(19) United States
(12) Patent Application Publication
(10) Pub. No.: US 2023/0404066 A1
(43) Pub. Date: Dec. 21, 2023

(54) TISSUE PERFUSION AND VIABILITY SENSING SYSTEM

(71) Applicant: Vascular Perfusion Solutions, Inc., San Antonio, TX (US)

(72) Inventors: Israel Jessop, Garden Ride, TX (US); Exal Cisneros, San Antonio, TX (US); Leonid Bunegin, San Antonio, TX (US)

(21) Appl. No.: 18/335,346

(22) Filed: Jun. 15, 2023

Related U.S. Application Data
(60) Provisional application No. 63/366,441, filed on Jun. 15, 2022.

(51) Int. Cl. A01N 1/02; A61M 60/31

(52) U.S. Cl. CPC A01N 1/0247; A61M 60/31

(57) ABSTRACT

A perfusion system includes a perfusate source and a perfusion distributor coupled to the perfusate source and configured to convey oxygen containing perfusate from the perfusate source to tissue and exhaust carbon dioxide generated by the tissue. A carbon dioxide sensor is coupled to sense the carbon dioxide generated by the tissue, which can then be used to provide a measure of tissue viability.

### RELATED APPLICATION

This application claims priority to U.S. Provisional Application Ser. No. 63/366,441 (entitled TISSUE PERFUSION AND VIABILITY SENSING SYSTEM filed Jun. 15, 2022) which is incorporated herein by reference.

### BACKGROUND

Perfusion includes the passage of fluid through the circulatory system or lymphatic system of an organ or tissue. In the human body, perfusion often refers to passage of blood through a capillary bed in tissue. Perfusion can allow for the delivery of oxygen, other dissolved gases, nutrients, and other items to the tissue. When tissue or an organ is not residing in the body, such as during transport of an organ for transplant, perfusion does not naturally occur, and this can result in unwanted damage to the tissue or organ.

### SUMMARY OF THE DISCLOSURE

A perfusion system includes a perfusate source and a perfusion distributor coupled to the perfusate source and configured to convey oxygen containing perfusate from the perfusate source to tissue and exhaust carbon dioxide generated by the tissue. A carbon dioxide sensor is coupled to sense the carbon dioxide generated by the tissue.

In a further example, a perfusion system includes a gas exchanger to receive oxygen from an oxygen source. A perfusate circulation system is coupled to the gas exchanger and configured to convey oxygen containing perfusate from the gas exchanger to tissue and return carbon dioxide containing perfusate from the tissue to the gas exchanger. A carbon dioxide sensor is coupled to measure carbon dioxide generated by the tissue.

### BRIEF DESCRIPTION OF THE DRAWINGS

In the drawings, which are not necessarily drawn to scale, like numerals may describe similar components in different views. The drawings illustrate generally, by way of example, but not by way of limitation, various embodiments discussed in the present document.

FIG. 1A is a block diagram of an example perfusion system capable of measuring tissue viability.

FIG. 1B is block diagram of an example gas exchanger illustrating gas being exchanged between two fluid flows.

FIG. 2 is a block diagram of an alternative example perfusion system capable of measuring tissue viability.

FIG. 3 is a block diagram of a further example perfusion system capable of measuring tissue viability.

FIG. 4 is a block diagram of yet a further example system capable of measuring tissue viability.

FIG. 5 is a block diagram of a still further example perfusion system capable of measuring tissue viability.

FIG. 6 is a block diagram of another example perfusion system capable of measuring tissue viability.

FIG. 7 is a flow diagram illustrating a method of determining ex-vivo tissue viability.

FIGS. 8A, 8B, and 8C are block diagrams of a perfusion system in an example.

### DETAILED DESCRIPTION

A tissue perfusion system is used to maintain the viability of an organ or other tissue for transplant following removal from a body and during a transport interval. The system provides perfusate for maintaining the viability of the organ or other tissue that is waiting to be given to a transplant recipient. The system can maintain and prolong organ viability during the transport interval, after removal from a donor but before transplantation into an organ recipient. As an alternative to placing the tissue in cold static storage, the tissue perfusion system can extend the time that tissue remains viable for transplant.

Once separated from a living body, organs, limbs, and other vascularized tissues may be oxygenated, and metabolic waste products, including carbon dioxide, removed. The organ perfusion system can prolong organ viability outside the body. Perfusion systems can pump an oxygen-enriched liquid through the vasculature (e.g., arteries, capillaries, and veins) of tissue. Moreover, perfusion can deliver nutrient gas, such as oxygen, and metabolic substrates, such as glucose, to metabolically active cells and simultaneously remove metabolic waste gas, such as carbon dioxide.

Vascularized tissue, hereinafter tissue, can be perfused during transport or transplant processes, research, and diagnostics, and other ex-vivo organ treatments. While the use of an organ perfusion system can extend the time that separated tissue remains viable, it can be difficult to measure the tissue viability in such systems.

An improved tissue perfusion system incorporates a carbon dioxide (CO2) sensor to measure the amount of CO2 added to a perfusate by tissue being perfused. Combined with knowledge of the perfusate or gas (as appropriate) rate of flow, the amount of CO2 produced by the tissue may be determined. By comparing the amount of CO2 produced to a theoretical amount the tissue should produce, an overall viability of the tissue being preserved by perfusion system can be determined.

CO2 is a product of aerobic cellular respiration in live animal tissue, where the cell breaks down macronutrients (such as proteins, carbohydrates, or fats) with the help of oxygen to replenish its energy reserves. As a corollary, once the cell dies such as by trauma or hypoxic conditions, it no longer performs cellular respiration and doesn't produce CO2 even in the presence of oxygen. A tissue having dead cells may no longer be viable/able to perform its functions if it were to be retransplanted. Therefore, knowing the CO2 production levels in relation to tissue weight can give an indication of amount of tissue that is alive and thus of the viability of the tissue.

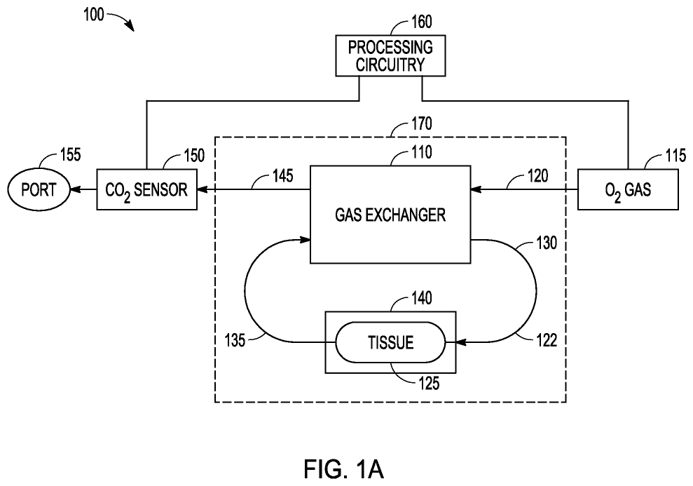

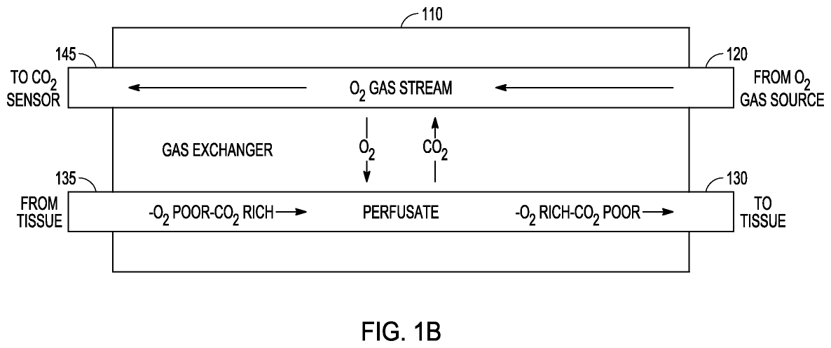

FIG. 1A is a block diagram of an example perfusion system 100 capable of measuring tissue viability. Perfusion system 100 includes a gas exchanger 110 coupled to an oxygen-rich gas source 115. The gas exchanger 110 receives gas containing oxygen via a conduit 120 and the gas exchanger 110 is used to oxygenate a perfusate 122. Example gas exchangers include a Medtronic AFFINITY NT oxygenator that is compatible with blood, and/or Permselect part number PDMSXA-1.0. The perfusate 122 may be a liquid in one example and is circulated via a perfusate circulation system 170, which is composed of the gas exchanger 110, conduits 130 and 135, and, if included, container 140, all used to perfuse the tissue 125 which may be coupled to the perfusate circulation system 170. The perfusate circulation system 170 may also include a pump as shown in later figures. Alternatively, where the tissue comprises a heart, the heart may serve as a pump to move fluid through the perfusate circulation system 170.

The perfusate 122 is coupled to a tissue 125 via perfusate supply conduit 130 of the perfusate circulation system 170 and may be pumped, impelled, or otherwise caused to move or flow to the tissue 125. The perfusate 122 oxygenates the tissue 125 and receives at least CO2 from the tissue 125.

The perfusate 122 exits the tissue 125 via an outlet conduit 135 of the perfusate circulation system 170. Said conduit couples the tissue 125 and gas exchanger 110 to return the perfusate 122 to the gas exchanger 110. The CO2 in the returned perfusate is removed from the perfusate at the gas exchanger 110. This occurs by the CO2 diffusing from the perfusate, across the gas exchanger's 110 membrane, into the oxygen-rich and CO2-poor gas coming from the oxygen-rich gas source 115 thru conduit 120 as shown in FIG. 1B. Likewise, the perfusate 122 is re-oxygenated by oxygen diffusing across the membrane from the oxygen-rich and CO2-poor gas stream into the perfusate 122 at the gas exchanger 110, returning to the tissue via supply conduit 130. The tissue may be in a sealed tissue canister 140 to prevent contamination and support the tissue during perfusion. The conduits may pass through walls of the canister 140 in a sealed manner.

The CO2 removed from the tissue in the perfusate 122 may be in a gaseous dissolved state or in the form of other chemical species that readily and reversibly dissociate into CO2, but exits the gas exchanger 110 as gas via an exhaust conduit 145. A CO2 sensor 150 is positioned to sense concentration of CO2 in the exhaust conduit 145 downstream of the gas exchanger 110. In one example, the CO2 sensor 150 may comprise a capnograph (non-dispersive infrared (NTRD) measuring technology) based type of CO2 sensor, such as one available from Philips Respironics: CAPNOSTAT 5 CO2 Sensor (Part Number: 989805618431).

Following measurement of the CO2, the gas in exhaust conduit 145 exits to the ambient environment at port 155. Processing circuitry 160 may be coupled to the CO2 sensor and receive data, an electrical signal, or some other indication representative of the amount of carbon dioxide in the gas exiting the gas exchanger 110 via exhaust conduit 145.

In one example, the flow rate of the gas from oxygen-rich gas source 115 is indicated to or stored in the circuitry 160, as is the mass of the tissue 125. The flow rate may be controlled, such as by a flow regulator, to a selected set point by circuitry 160 in one example or measured by an optional flow rate sensor. In addition, the gas from oxygen-rich gas source 115 may contain little if any CO2, and the perfusate 122 returned from the tissue 125 have waste CO2 removed by the appropriately sized gas exchanger 110 such that most of the CO2 produced by the tissue exits through the exhaust conduit 145 to contact the CO2 sensor 150.

The mass of the tissue 125 may be measured prior to perfusing the tissue 125 in one example. The mass of the tissue 125 may be derived from a change in weight of the system 100 after adding the tissue 125 or coupling the tissue 125 to the system 100. By knowing the mass, species, and metabolic state of the tissue, the expected rate of CO2 production for a given tissue mass can be determined. The expected rate of CO2 production, combined with the CO2 concentration and gas flow rate stored indications, can be used by circuitry 160 to calculate the proportion of the tissue that is alive and metabolizing, providing an indication of the viability of the tissue 125 for use, such as for a transplant. In further examples, a type of metabolism, such as aerobic, a type of macronutrient used, such as carbohydrates, fats, proteins and other types of nutrients used by the tissue may also be considered in determining the expected rate of CO2 production for a given tissue mass.

The following formula [1] may be used for calculating a rate of CO2 production by tissue 125, here Q_{CO2} is the rate of CO2 produced (if positive) or consumed (if negative), [CO2]_{IN} is the concentration of CO2 in all its species going in to the gas exchanger's gas side, [CO2]_{OUT} is the concentration of CO2 in all its species going out of the gas exchanger's gas side, and Q_{gas} is the volumetric gas flow across the gas exchanger.

Q_{CO2}={[CO₂]ₒᵤₜ−[CO₂]ᵢₙ}*Q_{gas} [1]

One example of application of formula [1] includes the scenario of a 0.200 L/min gas stream of 100% O2, where the CO2 concentration is known to be 0 ppm (parts per million), going into the gas exchanger 110 with no leakage in the gas exchanger 110. Coming out of the gas exchanger, the CO2 concentration is measured at 50,000 ppm by carbon dioxide sensor 150. The change in concentration is thus determined to be 50,000 ppm CO2 gas. Since ppm is a ratio of gases (e.g., of 1 million gas molecules, 50,000 (5%) would be carbon dioxide molecules), CO2 rate of production is obtained by multiplying the gas flow across the gas exchanger 110 by this ratio (50,000/1,000,000), which gives a CO2 production value of: 0.01 L CO2/min.

FIG. 1B is block diagram of an example gas exchanger 110 illustrating gas being exchanged between two fluid flows. A first fluid flow is via conduit 120 from oxygen-rich gas source 115. Conduit 120 proceeds through the gas exchanger and exits the gas exchanger as exhaust conduit 145. The second fluid flow is via conduits 135 and 130. In operation, the gas exchanger 110 passes oxygen from the oxygen-rich gas stream in conduit 120 to the supply conduit 130 for perfusion of the tissue 125. CO2 generated by the tissue 125 is returned to the gas exchanger 110 via inlet conduit 135. The gas exchanger 110 passes CO2 back from the CO2-rich perfusate coming from outlet conduit 135 to the gas stream exiting thru exhaust conduit 145 for sensing by the CO2 sensor.

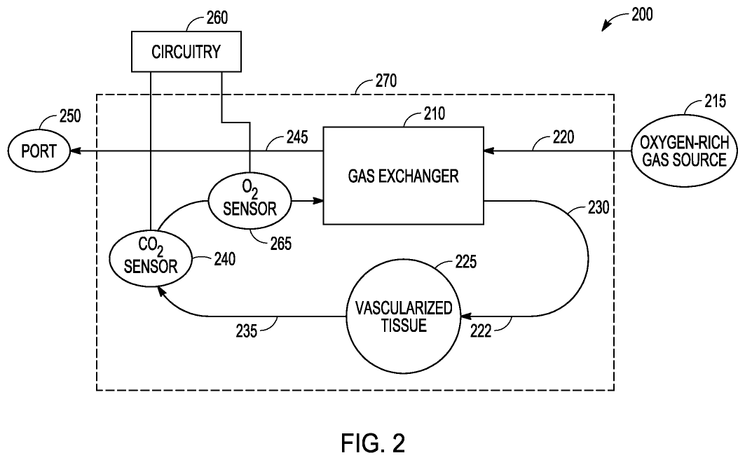

FIG. 2 is a block diagram of an alternative example perfusion system 200 capable of measuring tissue viability. Perfusion system 200 includes a gas exchanger 210 coupled to an oxygen-rich gas source 215. The gas exchanger 210 receives gas containing oxygen via a conduit 220 and is used to oxygenate a perfusate 222. The perfusate 222 may be a liquid in one example and is circulated via a perfusate circulation system 270. The perfusate 222 is coupled to tissue 225 via perfusate supply conduit 230 of the perfusate circulation system 270 and may be pumped or impelled to the tissue 225. The perfusate 222 oxygenates the tissue 225 and receives at least CO2 from the tissue. The perfusate circulation system 270 is thus composed of the gas exchanger 210 and conduits 230 and 235, all used to perfuse the tissue 225 which may be coupled to the perfusate circulation system 270.

The perfusate 222 exits the tissue 225 via an outlet conduit 235 of the perfusate circulation system 270 coupled between the tissue 225 and gas exchanger 210 to return the perfusate 222 to the gas exchanger 210. The CO2 in the returned perfusate 222 may be sensed via a dissolved CO2 sensor 240 coupled to the outlet conduit 235 between the tissue 225 and the gas exchanger 210. The sensor 240 is used to sense partial pressure of dissolved CO2 in the perfusate 222 downstream of the tissue 225, which combined with knowledge of the perfusate's composition (aqueous or not, presence of buffer system, CO2 carriers) may be used to determine CO2 concentration in the perfusate.

Dissolved CO2 sensor 240 in one example is an electrochemical sensor, such as a sensor utilizing a Severinghaus electrode. Example sensors include a sampling sensor, such one available from Abbot—i-STAT CG8+(Product Code 03P88-25.) Another example sensor utilizes in-line measurement, such as a CO2 Sensor InPro 5000i/12/120 available from Mettler Toledo.

Following measurement of the CO2 in the perfusate 222, the gas exchanger 210 removes CO2 from the perfusate 222 and reoxygenates the perfusate 222 which is recirculated back to the tissue 225 via the perfusate circulation system 270.

The removed CO2 is in a gaseous state and exits the gas exchanger 210 as gas via an exhaust conduit 245 where it exits to the ambient environment at port 250. Processing circuitry 260 may be coupled to receive data from the CO2 sensor 240 representative of the amount of CO2 in the perfusate 222 exiting the tissue 225.

In one example, the flow rate of the perfusate going across the perfusate circulation system is indicated or stored in the circuitry 260, as is the mass of the tissue 225. The flow rate may be controlled to a selected set point by circuitry 260 in one example or measured by an optional flow rate sensor. In addition, the gas from oxygen-rich gas source 215 may contain little if any CO2, and the perfusate 222 returned from the tissue 225 has waste CO2 removed by the appropriately sized gas exchanger 210 such that most of the CO2 produced by the tissue exits to the ambient environment via exhaust conduit 245 and the port 250.

The mass of the tissue 225 may be measured prior to perfusing the tissue in one example. The mass of the tissue 225 may be derived from a change in weight of the system after adding the tissue 225 or coupling to the tissue 225. By knowing the rates of CO2 utilization for a given tissue mass given its species and metabolic state, the amount of live and metabolizing tissue may be calculated, providing an indication of the viability of the tissue 225 for use, such as for a transplant. In further examples, a type of metabolism, such as aerobic, a type of macronutrient used, such as carbohydrates, fats, proteins and other types of nutrients used by the tissue may also be considered in determining the expected rate of CO2 production for a given tissue mass.

System 200 may also include an oxygen (O2) sensor 265 positioned to measure dissolved oxygen concentration in outlet conduit 235. Processing circuitry 260 may be coupled to receive data from the O2 sensor 265 representative of the amount of O2 in the perfusate 222 exiting the tissue 225. One example oxygen sensor 265 includes an optically based sensor, such as a PyroScience: Robust Oxygen probe OXROB10. In one example, the CO2 utilization is indicative of the proportion of tissue that is still alive, utilizing oxygen and producing CO2. In a further example, the measurements of CO2 and oxygen may be used by the circuitry 260 to determine a tissue's respiratory quotient (RQ), which is a ratio of CO2 released by tissue 225 divided by the oxygen absorbed by the tissue 225.

The same formula [1] used for CO2 mass production may be used to calculate the amount of O2 consumed and CO2 produced by the tissue, substituting the terms of CO2 concentration coming out of the gas exchanger ([CO₂]ₒᵤₜ) for the known and measured O2 and/or CO2 concentrations coming out of the tissue 225, while substituting the terms of CO2 coming into the gas exchanger ([CO₂]ᵢₙ) for the known and/or measured concentrations of O2 and/or CO2 coming out of the gas exchanger. Lacking sensors to measure the CO2 and/or O2 concentrations in perfusate 222 coming out of the gas exchanger, it can be assumed that the perfusate's 222 dissolved gases equilibrated completely with the sweep gas passing through the appropriately sized gas exchanger 210. With this assumption, the gas concentrations coming out of the gas exchanger 210 through conduit 230 and the into the tissue 225 can be known to be proportionally equal to those in the gas stream going into the gas exchanger. Note that the concentrations terms in formula [1] involve transport in all different species of Oxygen and carbon dioxide and not just the dissolved partial pressures, which may be the only reading of the respective sensors 265 and 240. One can use known properties of the perfusate 222, such as buffering systems or presence of gas carriers, to correlate the dissolved gas concentrations readings to total concentration of O2 and CO2 in all its species,

The RQ is obtained by dividing the amount of CO2 produced by the tissue by the amount of Oxygen consumed by the tissue in question. RQ helps identify what type of substrate the tissue in question is metabolizing to get its energy. By knowing what type of macronutrient the tissue is metabolizing, a more precise measure of how much energy the tissue is consuming may be calculated.

$$Respiratory Quotient ( RQ ) = \frac{Δ {M}_{C O 2}}{Δ {M}_{O 2}} \qquad [ 2 ]$$

Where RQ is the respiratory quotient to be calculated, ΔM_{CO2} is the amount of CO produced by the tissue, and ΔM_{O2} is the amount of oxygen consumed by the tissue.

According to Hiran Patel et. al in “Phisiology, Respiratory Quotient”, StatPearls, typical RQ values for macronutrients in one example include Carbohydrate RQ=1, Protein RQ=0.8, and Fats RQ=0.7. If the tissue is metabolizing a combination of these substrates, then the Respiratory Quotient RQ should hover around ˜0.8.

To use the amount of CO2 produced as a measure of viability of the tissue, an expected metabolic rate for the tissue should be established. Metabolic rate means the amount of energy consumed by the tissue over time. This value depends on several factors, including temperature, activity level of the tissue in question (for example, whether it is operating at its full capacity, or at some fraction of full capacity), type of tissue (heart, liver, kidney), and in some cases, the animal species that supplies the tissue. For example, the list below includes typical resting metabolic rates for human organs operating at body temperature, as is widely established in the scientific literature, summarized for an example in “Specific Metabolic Rates of Major Organs and Tissues Across Adulthood: Evaluation by Mechanistic Model of Resting Energy Expenditure” by ZiMian Wang and et. al (2010):
Heart: 440 kcal/kg/day
Kidney: 440 kcal/kg/day
Liver: 200 kcal/kg/day

Next, theoretical energy consumption is converted to a corresponding production of CO2. For this, it is helpful to know the type of macronutrient used by the tissue, since this affects the amount of energy released for a given volume of CO2 as shown in Table 1.

TABLE 1
Energy release per unit volume of O₂ and
CO₂ and RQ for metabolism of different macronutrients,
obtained from “METHOD OF CALCULATING THE
ENERGY METABOLISM”, Acta Pediatrica (1952)

| | Caloric value | | |
|---|---|---|---|
| | O₂ | CO₂ | |
| Substance | Cals./lit. | Cals./lit. | RQ |
| Carbohydrate | 5.047 | 5.047 | 1.000 |
| Fat | 4.686 | 6.629 | 0.707 |
| Protein | 4.485 | 5.599 | 0.801 |

Knowing the RQ of the tissue, it is possible to identify which macronutrient might be used and more accurately estimate the ratio of CO2 produced to energy consumption. For example, if the RQ of a given tissue is measured to be 0.7, then referring to Table 1 above, it can be deduced that the organ is only metabolizing fat, and thus, 6.629 kcal of energy consumed for every liter of CO2 consumed by the tissue. If the oxygen consumption value is not available, and thus RQ value not able to be determined, it is possible to estimate the amount of tissue energy consumption for a given CO2 production by noting the maximum and minimum energy production for any given macronutrient. For example, according to Table 1, for a given liter of CO2 the lowest amount of energy consumed would come from metabolizing fat carbohydrates at 5.047 kcal/L CO2, while the highest amount of energy would come from metabolizing fat at 6.629 kcal/L CO2. Therefore, one can expect the energy consumption for any given macronutrient consumed by a tissue to be between 5.047 and 6.629 kcal/L CO2.

Assuming the type of macronutient being metabolized is known (be this from RQ value or other indication), it is possible to determine the energy consumption to CO2 production ratio (K_{macronutrient,CO}_{₂}) for the given macronutrient as explained above. Further, knowing the mass of the tissue (Mₜᵢₛₛᵤₑ), and the basal metabolic rate of the tissue at the given preservation conditions (Kₜᵢₛₛᵤₑ), then it is possible to use formula [3] below to calculate the theoretical amount of CO2 (Q_{CO}_{₂}_{_theoretical}) the tissue should be producing in order to satisfy its metabolic demands and remain viable.

$$\frac{{K}_{tissue} * {M}_{tissue}}{{K}_{macronutrient , {CO}_{2}}} = {Q}_{C {O}_{2} \_ theorethical} \qquad [ 3 ]$$

In one example, a 300 g human heart is preserved in a working (beating) state at body temperature, with in an expected metabolic rate of 0.306 kcal/kg/min (see paragraph [39] above). The RQ is not known, and thus the 5.047 to 6.629 kcal/L CO2 macronutrient conversion factor range will be used. This results in the tissue needing to produce somewhere between 0.014 and 0.018 L CO2/min to satisfy its metabolic demands. If we were to use the CO2 production value 0.01 L CO2/min from the ‘CO2 mass production’ example above (paragraph 26), this may indicate that the organ is not being fully perfused, or that some portion of the tissue is no longer living or viable.

By doing the above calculation, it is possible to provide the transplant doctor with a quantitative measure of tissue viability. In this case, tissue viability is defined as the proportion of the tissue being preserved that is still alive and metabolizing. The transplant doctor could then use this metric to gauge whether the tissue would be able to perform its functions when transplanted back into the body. For example, the “CO2 mass production” example indicated a CO2 production of 0.01 L CO2/min. However, our calculations above indicate that this tissue should have been producing between 0.014 and 0.018 L CO2/min to satisfy its metabolic demands. Formula [4] below, can be used to calculate the viability of the tissue being preserved, where Q_{CO2_experimental} is the measured amount of CO2 produced (calculated using formula [1] above) and Q_{CO}_{₂}_{_theoretical} is obtained from formula [3] above. By using formula [4] we can then calculate the tissue viability of the tissue producing 0.01 L CO2/min to be 70% for the lower range (0.014 L CO2/min) and 55% for the upper range (0.018 L CO2/min) of said required theoretical consumption. The transplant doctor can then determine whether he/she believes this tissue viability percentage to be sufficiently high to ensure the tissue will be able to fulfill its function when reimplanted into the body. The determination may be based on studies, experience, or a combination of both.

$$Tissue Viability ( \% ) = \frac{{Q}_{CO 2 \_ experimental}}{{Q}_{C {O}_{2} \_ theorethical}} \qquad [ 4 ]$$

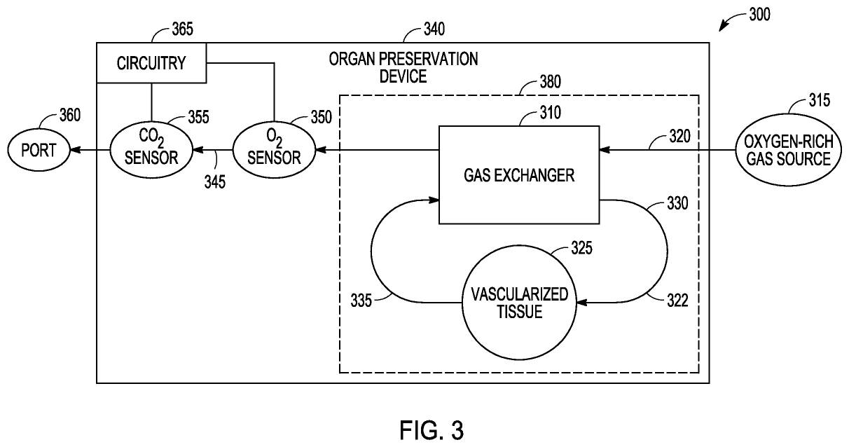

FIG. 3 is a block diagram of a further-example perfusion system 300 capable of measuring tissue viability. Perfusion system 300 includes a gas exchanger 310 coupled to an oxygen-rich gas source 315. The gas exchanger 310 receives gas containing oxygen via a conduit 320 and is used to oxygenate a perfusate 322. The perfusate 322 may be a liquid in one example and is circulated via a perfusate circulation system 380. The perfusate 322 is coupled to a tissue 325 via perfusate supply conduit 330 of the perfusate circulation system 380 and may be pumped or impelled to the tissue 325. The perfusate 322 oxygenates the tissue 325 and receives at least CO2 from the tissue.

The perfusate 322 exits the tissue 325 via an outlet conduit 335 of the perfusate circulation system 380, coupled between the tissue 325 and gas exchanger 310 to return the perfusate 322 to the gas exchanger 310. The CO2 in the returned perfusate 322 is removed by diffusion across the gas exchanger, down its concentration gradient, from the perfusate 322 into the gas stream. The perfusate 322 is re-oxygenated by the gas exchanger 310 by diffusion of oxygen, down its concentration gradient, across the gas exchanger from the gas stream into the perfusate 322 and is returned to the tissue 325 via supply conduit 330. The tissue 325 and other elements included in the system 300 or coupled to the system 300 if desired, may be supported in a sealed canister to prevent contamination and support the tissue 325 during perfusion. The perfusate circulation system 380 thus includes the gas exchanger 310 and conduits 330 and 335 to perfuse the tissue 325 which may be coupled to the perfusate circulation system 380.

The removed CO2 may be in a gaseous state and exits the gas exchanger 310 as gas via an exhaust conduit 345. An oxygen sensor 350 and a CO2 sensor 355 may be positioned to sense oxygen and CO2 concentrations in the exhaust conduit 345 downstream of the gas exchanger 310. Example oxygen sensors include a heated current limiting sensor from ServoFlo: FCX-UWC, and/or an electrochemical-based oxygen sensor—Honeywell: MOX9 Medicel® Part #: AAD29-210.

Following measurement of the oxygen and CO2, the gas in exhaust conduit 345 exits to the ambient environment at port 360. Processing circuitry 365 may be coupled to receive data, an electrical signal, or some other indication representative of the amount of CO2 and/or O2 in the gas exiting the gas exchanger 310 via exhaust conduit 345.

In one example, the flow rate of the gas from oxygen-rich gas source 315 as well as the fraction of the gas composed of oxygen is known to the circuitry 360, as is the mass of the tissue 325. The flow rate may be controlled to a set point by circuitry 360 in one example or measured by an optional gas flow rate sensor. In addition, the gas from oxygen-rich gas source 315 may contain some, or no CO2, and the perfusate 322 returned from the tissue 325 has waste CO2 removed by the appropriately-sized gas exchanger 310 such that most or all of the CO2 produced by the tissue exits to the ambient environment via the exhaust conduit 345 and the port 360, after passing by the carbon CO2 sensor 355.

The mass of the tissue 325 may be measured prior to perfusing the tissue 325 in one example or derived from a change in weight of the system 300 after adding the tissue 325. By knowing the rate of CO2 utilization of a given tissue mass, the amount of live tissue may be calculated by circuitry 365 using formula [4], providing an indication of the viability of the tissue 325 for use, such as for a transplant. In other words, the CO2 production is indicative of the proportion of tissue that is still alive, utilizing oxygen and producing CO2. In a further example, the measurements of CO2 and oxygen may be used by the circuitry 365 to determine a tissue respiratory quotient, which is a ratio of CO2 released from the tissue 325, divided by the oxygen absorbed by the tissue 325 (formula [2]).

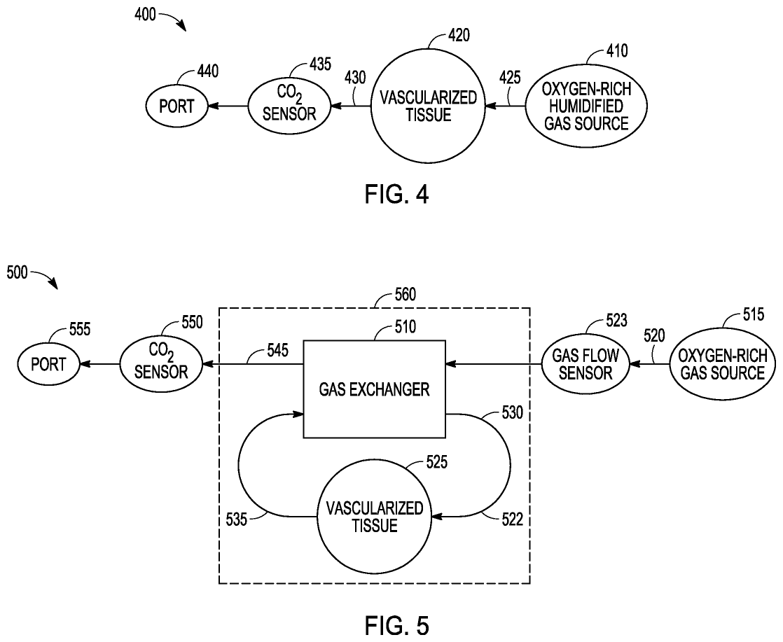

FIG. 4 is a block diagram of yet a further example system 400 capable of measuring tissue viability. Persufflation is an alternative to machine perfusion to deliver gaseous oxygen to tissue and remove gaseous CO2. By moving an oxygen-rich gas stream through the vasculature of the tissue 420, a liquid solution (i.e., as the perfusate) and gas exchanger may not be needed. Instead, the CO2 concentration of the gas stream is measured downstream of the tissue 420. For the purposes of this document, “oxygen-rich” means gas mixtures compromised of at least 150 mmHg oxygen partial pressure or whatever levels are required to maintain the tissue metabolic levels.

In system 400, an oxygen-rich humidified gas source 410 provides gaseous oxygen operating as an analogous perfusate 322 to oxygenate tissue 420 via a supply conduit 425. The gas source 410 may be humidified in a common manner, such as by using a humidification system that passes gas through a wet porous material, a bubbling water source, heated water, or other source of water vapor. The tissue 420 consumes oxygen and generates CO2. Remaining gas flow exits the tissue via an outlet conduit 430 that is coupled to a carbon dioxide sensor 435 for sensing CO2 concentration in the outlet conduit 430, before exiting thru port 440 to the ambient environment. As described above in previous figures, the CO2 concentration along with the known gas flow rate and known tissue mass may be used to determine tissue viability by suitable circuitry, such as a programmed processor. For the purposes of this document, a “humidified” gas source provides a humidity level of at least 95% in the stream of gas passing through the conduit 425. Other humidity levels may be used in further examples, such as less than 95% or more than 95%.

In a further example, the gas source 410 may be a perfusate fluid source, replacing the gas exchanger element. The perfusate may simply pass through the tissue via and exit through port 440 to the ambient environment or a collection vessel without being recirculated. In this example, the perfusate circulation system does not recirculate perfusate, but simply passes perfusate through the tissue 420.

FIG. 5 is a block diagram of a still further example perfusion system 500 capable of measuring tissue viability. Perfusion system 500 includes a gas exchanger 510 coupled to an oxygen-rich gas source 515. The gas exchanger 510 receives gas containing oxygen via a conduit 520. Conduit 520 may include a gas flow sensor 523 to measure gas flow rate. There are several types of gas flow sensors or meters that may be used, such as a rotameter (Maxtex P#: RP34P03-009), micro-electro mechanical system (MEMS) (ServoFlo Model FS6122), electronic mass flow meter (Omega: FMA4312-02), flow regulator that controls the flow rather than sensing the flow (WESTERN-MEDICAL-OPA-510), and/or heat transfer flow sensor (Honeywell: AWM3000.) The gas flow sensor 523 may be used in any of the examples herein and may also serve as a regulator to control the gas flow rate.

The oxygen coming from the oxygen-rich gas source 515 in the flowing gas is used to oxygenate a perfusate 522 at the gas exchanger 510. The perfusate 522 may be a liquid in one example and is circulated via a perfusate circulation system 560, composed of gas exchanger 510, conduits 530 and 535, and mechanism used to move the perfusate 522 along the circuit and perfuse tissue 525, which may be coupled to the perfusate circulation system 560. The perfusate 522 is coupled to a tissue 525 via perfusate supply conduit 530 of the perfusate circulation system and may be pumped or otherwise moved to the vasculature of the tissue 525. In this way, the perfusate 522 oxygenates the tissue 525 and receives at least CO2 from the tissue.

The perfusate 522 exits the tissue 525 via an outlet conduit 535 of the perfusate circulation system 560 coupled between the tissue 525 and gas exchanger 510 to return the perfusate 522 to the gas exchanger 510. The CO2 in the returned perfusate 522 is removed from the perfusate 522 by diffusion across the gas exchanger, down its concentration gradient, from the perfusate 522 into the gas stream. The perfusate 522 is re-oxygenated by the gas exchanger 510 and is returned to the tissue 525 via supply conduit 530.

The CO2 removed from the perfusate 522 by gas exchanger 510 may be in a gaseous state and exits the gas exchanger 510 as a gas via an exhaust conduit 545. A CO2 sensor 550 is positioned to sense CO2 in the exhaust conduit 545 downstream of the gas exchanger 510. Following measurement of the CO2, the gas in exhaust conduit 545 exits to the ambient environment at port 555.

In one example, the flow rate of the gas from oxygen-rich gas source 515 is measured by gas flow sensor 523. The mass of the tissue 525 is also known. In addition, the gas from oxygen-rich gas source 515 may contain little or no CO2, and the perfusate 522 returned from the tissue 525 has waste CO2 removed by the appropriately sized gas exchanger 510 such that most or all of the CO2 produced by the tissue exits to the ambient environment via exhaust conduit 545 and port 555, after having passed CO2 sensor 550.

The mass of the tissue 525 may be measured prior to perfusing the tissue in one example or derived from a change in weight of the system after adding the tissue 525. By knowing the rates of CO2 utilization of a given tissue mass, the amount of live tissue may be calculated, providing an indication of the viability of the tissue 525 for use, such as for a transplant.

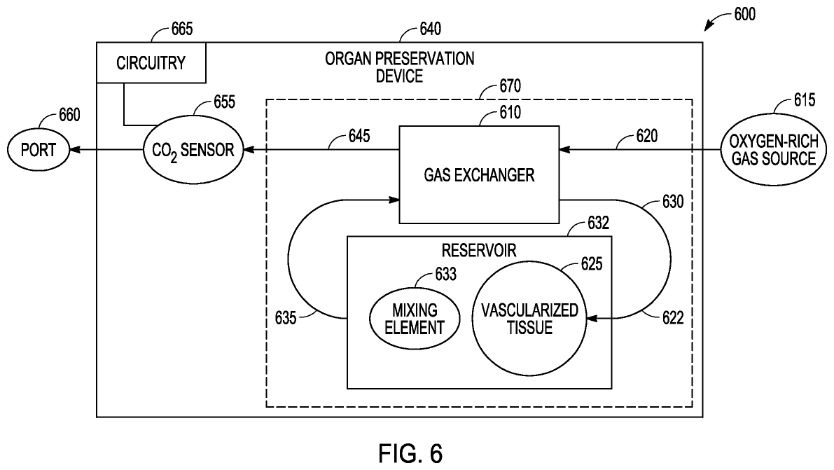

FIG. 6 is a block diagram of another example perfusion system 600 capable of measuring tissue viability. Perfusion system 600 includes a gas exchanger 610 coupled to an oxygen-rich gas source 615. The gas exchanger 610 receives gas containing oxygen via a conduit 620 and is used to oxygenate a perfusate 622. The perfusate 622 may be a liquid in one example and is circulated via a perfusate circulation system 670. The perfusate 622 is coupled to a tissue 625 via perfusate supply conduit 630 of the perfusate circulation system 670 and may be pumped or otherwise moved to the tissue 625. The perfusate 622 oxygenates the tissue 625 and receives at least CO2 from the tissue.

The perfusate 622 exits the tissue 625 into a perfusate reservoir 632 that encloses the tissue 625. The supply conduit 630 may be coupled to tissue 625 through a wall of the reservoir 632 in a sealed manner. The perfusate 622 exiting the tissue 625 is contained in the reservoir 632 and is mixed via a mixing element 633 which may provide mechanical agitation (e.g., a stirring bullet, a recirculating pump). In one example, the reservoir 632 may include a geometric design that promotes turbulent/mixing flow within the reservoir 632, or other means of mixing the perfusate 622 exiting the tissue 625 to ensure a substantially homogeneous concentration of CO2 exiting the reservoir 632 thru conduit 635. In one example the mixing element 633 is placed in a fairly large volume of perfusate 622 (e.g., the volume of the mixing element comprises less than 10% of the volume of the reservoir 632) in the reservoir 632 where the tissue 625 is submerged, so as to agitate the fluid in the reservoir 632 and achieve substantially uniform distribution of the CO2.

The perfusate 622 exits the reservoir 632 via an outlet conduit 635 of the perfusate circulation system coupled between the reservoir 632 and gas exchanger 610 to return the perfusate 622 to the gas exchanger 610. CO2 in the returned perfusate 622 is removed from the perfusate 622 at the gas exchanger by diffusion across the gas exchanger, down its concentration gradient, from the perfusate 622 into the gas stream. The perfusate 622 is re-oxygenated by the gas exchanger 610 by diffusion across the gas exchanger, down its concentration gradient, from the oxygen-rich gas stream into the perfusate 622, and is returned to the tissue 625 via supply conduit 630. The tissue 625 and other components may be arranged in a sealed canister 640 to prevent contamination and support the tissue 625 during perfusion. The perfusate circulation system 670 thus includes the gas exchanger 610, conduits 630 and 635, reservoir 632 and the optional mixing element 633, all used to perfuse tissue 625, which may be coupled to the perfusate circulation system 670.

The removed CO2 removed from the perfusate 622 at the gas exchanger may be in a gaseous state and exits the gas exchanger 610 as gas via an exhaust conduit 645. A CO2 sensor 655 may be positioned to sense CO2 concentrations in the exhaust conduit 645 downstream of the gas exchanger 610. Following measurement of the CO2, the gas in exhaust conduit 645 exits to the ambient environment, at port 660. Processing circuitry 665 may be coupled to receive data from CO2 sensor 655 representative of the amount of CO2 in the gas exiting the gas exchanger 610 via exhaust conduit 645. The data may be processed in the manner described in previous figures to determine tissue viability.

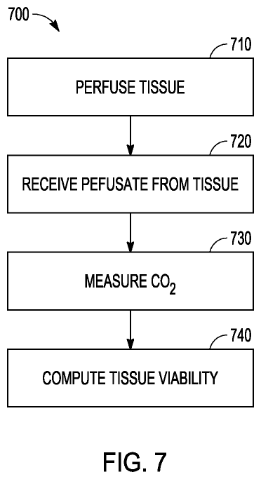

FIG. 7 is a flow diagram illustrating a method 700 of determining ex-vivo tissue viability. Method 700 includes, at operation 710, perfusing tissue with an oxygenated perfusate of known or measured CO2 concentration. The perfusate is received from the tissue at operation 720. Rate of CO2 generated by the tissue is measured at operation 730, and tissue viability is computed at 740 based on the measured CO2 production and tissue characteristics by using formula [4].

Measuring CO2 generated by the tissue in operation 730 may be performed by measuring change in CO2 concentration and multiplying the CO2 concentration change by a flow rate as explained in formula [1]. The measurement may be performed in the perfusate, which may be liquid (as part of a perfusion method) or gas (as part of a persufflation method), or in the gas stream across the gas exchanger in the perfusion method in various examples.

In a further example, method 700 includes measuring oxygen consumed by the tissue in parallel to operation 730 and calculating the RQ from the measured CO2 production and oxygen consumption, according to formula [2].

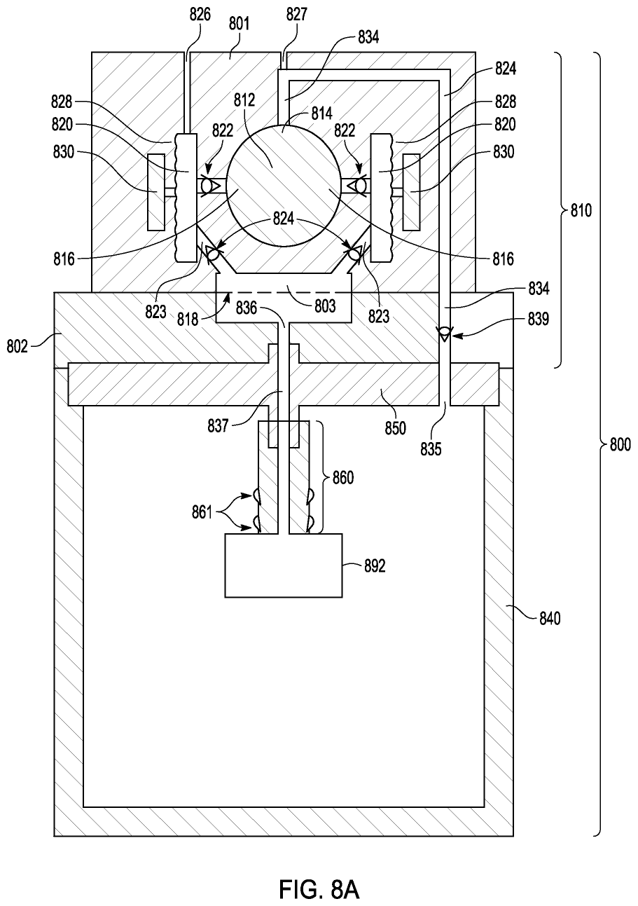

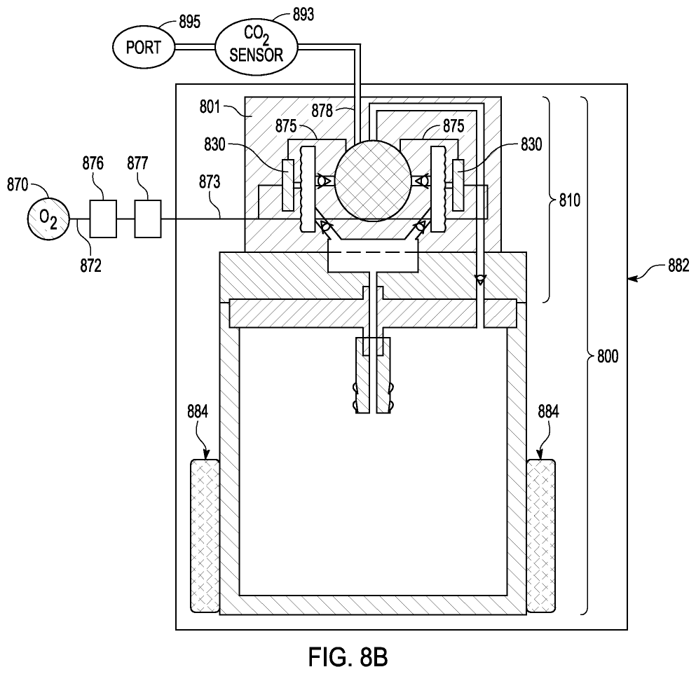

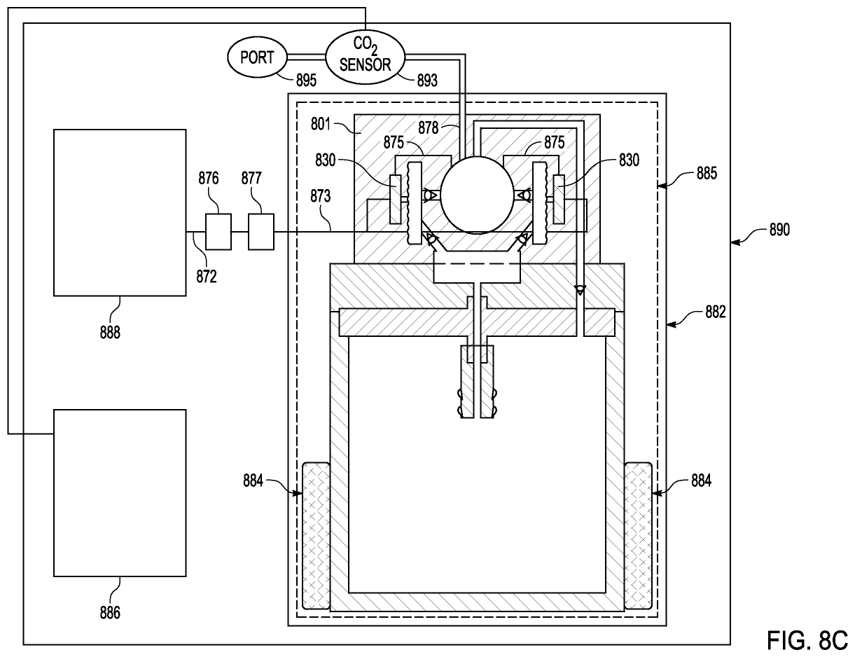

FIGS. 8A, 8B, and 8C illustrate a detailed block diagram of an example perfusion system 800. The system 800 can include a perfusion module 810, a tissue interface 850, and a canister 840. The perfusion module 810 can include pumps, valves, gas exchangers, filters, ports for fluid filling or extraction, sensors, fluid conduits, seals, a manifold 801 to connect the components, a base plate 802, and other components. The tissue interface 850 can include pass-through fluid channels, fluid port mating features, structural supports, and cannula attachment features. The canister 840 can include a perfusate fluid reservoir, mechanical fasteners, and in some cases, elements for thermal regulation of the system.

In the example system 800, the perfusion module 810 can contain a gas exchanger 812, a filter 818, pump chambers 820, and perfusate lines 834 and 836 coming from the canister 840 and going toward a cannula 860, respectively. In the embodiment shown, two pumps 820 are used in parallel. Other embodiments may utilize a single pump. Throughout the description, elements that are introduced as including one or more of an element may be referred to in the plural form for convenience without precluding examples that include only one of the elements.

In the perfusion module 800, the gas exchanger 812 can be disposed within the manifold 801. The gas exchanger 812 can include a perfusate inlet 814 and one or more outlets 816. In further examples, the pumps 820 may be positioned prior to the gas exchanger 812. The outlets 816 can provide oxygenated perfusate to one or more pump chambers 820. The filter 818 can be disposed within a filter chamber 803 at the junction between the manifold 801 and the base plate 802, the filter chamber may be comprised of cavities in the manifold 801 and/or the baseplate 802. The pump chambers 820 can be positioned to receive perfusate from the gas exchanger 812 via the one or more outlets 816. The pump chambers 820 can include inlet valves 822 positioned to control perfusate flow into the pump chambers 820 from gas exchanger outlets 816. The pump chambers 820 also can include outlets 823 with valves 824 to control perfusate flow out of the pump chamber into a filter chamber 803 which can connect to a perfusate supply line 836. A vent 826 can be connected to either or both pump chambers 820, pump outlets 823, or filter chamber 803 for venting gas. A vent 827 can connect to the perfusate line 834 for venting gas. The pump chambers 820 can include diaphragms 828 that are coupled to and controlled by valves 830. The diaphragms 828 can be actuated to pump perfusate through the perfusion module 810.

In the example system 800, the canister can be configured to hold an organ or tissue 892. The perfusate inlet opening 834 can be fluidically coupled to the canister 840.

In the example system 800, the tissue interface 850 can be positioned between the canister 840 and the base plate 802 of the perfusion module 810. The tissue interface 850 can include the perfusate inlet opening 835 that fluidly couples to perfusate line 834 and a perfusate line 837 that fluidly couples to perfusate line 836, to which a cannula 860 may be coupled. In one example, the tissue interface 850 simply provides a convenient connection between the cannula 860 and the perfusion module 810.

The cannula 860 can be hermetically sealed with the perfusate line 837 and configured with an end portion 861. The end portion 861 may be configured with one or more barbs or ribs also indicated at 861 to securely couple to an artery of a separated organ to supply oxygenated perfusate via perfusate supply line 836. The tissue interface can provide a secure, fluid tight, connection to canister 840 and base plate 802 while permitting controlled flow of perfusate to and from the canister. A one-way valve 839 can be included in perfusate line 834 to prevent retrograde flow of perfusate during priming.

As shown in FIG. 8B, the system 800 can be connected to an oxygen source 870 to supply oxygen via an oxygen line 872. The oxygen line 872 can be coupled to the pressure regulator 876 to regulate the oxygen pressure. The regulator 876 may also be connected to a flow controller or flow restrictor 877 to control or restrict flow of oxygen-rich gas to the system 800. An oxygen supply line 873 can extend from the pump pressure regulator 876 (or flow controller 877 if used) to the valves 830. One or more oxygen supply lines 875 can extend from the valves 830 to the gas exchanger 812 to provide oxygen for oxygenation of the perfusate. In one example, the oxygen supply lines 875 can extend or feed through the manifold 801 to reach the gas exchanger 812 to ensure proper fluidic sealing of gas exchanger 812. The manifold 801 can also include a vent 878 extending from the gas exchanger to an port 895 exposed to the ambient environment.

In some cases, such as shown in FIG. 8C, accessories such as a thermal barrier 882, phase change materials 884, electronics module 886, oxygen tank 888, and a carry case 890, can be included.

In one example, as in the example perfusion system 100 shown in FIG. 1A, the vent 878 is coupled to a CO2 sensor 893 before going to port 895 going to ambient environment. In this case, the CO2 sensor could send an indication to the electronics module 886. The electronics module 886 could serve here the purpose of circuitry 160 in FIG. 1, where it uses said indication of CO2 concentration and combines it with an indication of mass of the tissue 892, metabolic state of the tissue, and gas flow rate indication across the gas exchanger 812, such as from the flow regulator 876, to provide a viability indication of the tissue by using formula [4]. In this case, the perfusion system 800, would represent the perfusion circulation system 100 in FIG. 1,

In the example of FIGS. 8A-8C, the system 800 can be used to circulate perfusate through a target tissue or organ in the canister 840 to provide oxygen to the tissue. The perfusate can be a perfusate fluid, such a liquid, blood, saline, fluid specifically formulated for organ preservation or perfusion, or some other appropriate fluid for perfusion of an organ or target tissue in the canister 840 such as the humidified gas previously described for persufflation. The perfusate fluid can include water, electrolytes, pH buffering components, metabolic substrates, and other ingredients to maintain the viability of the heart. The fluid can be, for example, oxygen-enriched fluid or blood-based fluid, or humidified gas to provide oxygen to the target tissue, organ, or limb. For example, the organ can be a heart, lung, kidney, or other vascular tissue requiring oxygenation while outside the body. The perfusion circuit can include, for example, tubing, piping, or hosing, to carry the perfusate fluid between one or more fluid reservoirs, and the canister 840.

The perfusion module 810 can include pumps, valves, gas exchangers, filters (as previously described), ports for fluid filling or extraction, sensors (such as previously described with respect to FIGS. 1-6), fluid conduits, seals, and other components. In the example system 800, the perfusion module 810 can house components for circulation of perfusate and oxygen throughout the system 800. The perfusion module 810 can include the gas exchanger 812, the filter 818, and the perfusion pump chambers 820, encapsulated by an optional housing (not shown) or a manifold 801. The perfusion module 810 can be connected to the canister 840, such as through the tissue interface 850. The cannula 860 may fluidly connect the perfusion module 810 to a cannulated organ or target tissue 892 located in the canister 840 by allowing flow of perfusate therebetween. The oxygen source 870 (FIG. 8B) or 888 (FIG. 8C) can be in fluid communication with the perfusion module 810 to allow flow of oxygen and allow for pressurization of the pump diaphragms 828.

The gas exchanger 812 can be configured as described above to exchange oxygen and CO2 in perfusate fluid. The gas exchanger 812 can include a perfusate inlet 814 for incoming de-oxygenated perfusate from the canister 840, and perfusate outlets 816, wherein outgoing oxygenated perfusate can exit the gas exchanger 812. The gas exchanger 812 can be secured within the perfusion module 810, such as to a base plate 802, or within a manifold 801.

The gas exchanger can be fluidly coupled to the oxygen source 870 (as in FIG. 8B) or 888 in FIG. 8C. The oxygen source 870 (FIG. 8B) or 888 (FIG. C) can be an oxygen concentrator, an oxygen generator, tank of pressurized oxygen, or other appropriate oxygen source, such as a hook-up. The oxygen source 870 (FIG. 8B) or 888 (FIG. C) can provide oxygen to the organ preservation system 800 and provide a pressure gradient to the system 800 to induce flow of a perfusate fluid therethrough. The oxygen source 870 (FIG. 8B) or 888 (FIG. C) may also supply oxygen in mixture with other gases, such as with carbogen (95% oxygen/5% CO2), oxygen/nitrous oxide mixtures, oxygen/hydrogen mixtures, etc.

For example, the oxygen source 870 (FIG. 8B) or 888 (FIG. C) can be an oxygen concentrator that can filter surrounding air, compress that air to a specified density, and deliver purified oxygen in a pulsatile fashion, or in a continuous stream. Such an oxygen concentrator can be fitted with filters and/or sieve beds to remove nitrogen and other elements, gases, or contaminants from the air. In an example, the oxygen concentrator can include a pressure swing adsorption system, such as the Invacare® Platinum Mobile oxygen concentrator (Invacare Corporation, Elyria, OH). A pressure swing adsorption oxygen concentrator can leverage a molecular sieve to absorb gases and operate using rapid pressure swing adsorption to capture atmospheric nitrogen in minerals, such as zeolite, and subsequently vent that nitrogen, operating in a manner that is similar to a nitrogen scrubber. This can allow other atmospheric gases to exit the system, leaving oxygen as the primary remaining gas. Conventional oxygen concentrators can include an air compressor, the molecular sieve or alternatively a membrane, a pressure equalizer, and various valves and tubes to accomplish these functions. Other types or configurations of oxygen concentrators or oxygen sources are also envisioned herein.

The pressure of the oxygen provided by the oxygen source 870 (FIG. 8B) or 888 (FIG. C) can be regulated by pump pressure regulator 876. The pressure can be about, for example, 75 mm Hg. Also waste gas can be vented out of the oxygenator from vent 878 to port 895 going to ambient environment.

The filter 818 can be, for example, a plate filter across the junction of the manifold and base plate 802, so that oxygenated perfusate leaving the pump chambers 820 can be filtered for impurities before being cycled back towards the cannula (860) and attached organ or tissue 892. The filter chamber 803 is formed by the combination of a cavities in the manifold 801 and base plate 802 where they come together.

The filter can include, for example, a particulate filter, a filter for removing contaminants in the perfusate fluid, a filter directed to chemicals or dissolved gases, or any other type of appropriate filter for treatment of the perfusate fluid. In any example of the portable oxygen source and perfusion system disclosed herein, multiple filters can be used. In some cases, a filter can be upstream of the tissue container 840 of the organ preservation system 800 so as to filter the perfusate fluid prior to reaching the tissue or organ 892 being perfused. In some cases, the filter can be downstream of the tissue container 840 of the organ preservation system 800 so that fluid returning to the tissue container reservoir is filtered.

The oxygenated perfusate can flow out of the oxygenator 812 through the valves 822 into the pump chambers 820. The pump chambers 820 can have inlet valves 822 and outlet valves 824, which can be check valves. The diaphragms 828 in the pump chambers 820 can be de-pressurized to allow flow of the oxygenated perfusate into the pump chamber 820s as the diaphragms relax. The oxygenated perfusate can flow into the pump chambers 820 through the inlet valves 822 and fill the pump chamber 820s partially or fully. The oxygenated perfusate can remain in the pump chambers 820 until it is pumped out towards the filter 818 and cannula 860.

The diaphragms 828, located in the pump chambers 820, can be pressurized to pump perfusate out of the pump chambers 820 through the outlet valves 824 as the diaphragms distend, and towards the target tissue in the canister 840 via line 836. Distension of the diaphragms 828 can allow pumping of the perfusate out of the pump chambers 820.

The valves 830 may be 3-way controllable solenoid valves situated in the oxygen line 873 between the oxygen pressure regulator 876 (or flow restrictor/regulator 877) and the oxygenator 812. The valve 830 is also between line 873 and diaphragms 828. Valves 830 may be fluidly coupled to the diaphragms 828.

The canister 840 can include a perfusate fluid reservoir, mechanical fasteners, and in some cases, elements for thermal regulation of the system. In the example of system 800, the canister 840 can be a container for the target tissue or organ being perfused. For example, the canister 840 can contain the perfusate and a heart (or other organ or tissue), coupling with the perfusion module 810 to form a sterile barrier around the organ, enclosing it within a fluid-tight container. The canister 840 can provide a sterile environment in which to transport and perfuse the target tissue and organ; the canister 840 can be filled with a perfusate in which the target tissue or organ resides.

The tissue interface 850 can include pass-through fluid channels, fluid port mating features, structural supports, and cannula attachment features. In the example system 800, the canister 840 can create a seal with the tissue interface 850 and be fluidly connected to the components of the perfusion module 810 through the cannula 860 and the tissue interface 850. The seal can be created by attachment mechanisms, such as threading, a snap fit, a press fit, O-rings, or other sealing attachments to allow for a liquid-tight seal. In the example system 800, the perfusion module 810 can be held in place atop the canister 840, such as by buckles or latches.

The tissue interface 850 between the canister 840 and the perfusion module 810 can separate the two. The tissue interface 850 can additionally mediate fluid transport between the perfusion module 810 and the target tissue or organ, and back into the perfusion module 810.

The cannula 860 can fluidly connect the perfusion module 810 to the target tissue 892 through the tissue interface 850. For example, where a heart is being transported and perfused, the cannula 860 can fluidly couple the aorta of the donor heart to the output of the perfusion module 810, and also support the weight of the donor heart during transfer to the sterile surgical field.

In some cases, such as shown in FIG. 8B, accessories such as a thermal barrier 882 configured to enclose the system 800 to prevent heat transfer to and from an ambient environment may be included. Phase change materials 884 can be coupled to container 840. An electronics module 886 and, oxygen tank 888 and thermal barrier 882 may be disposed or otherwise supported within a carry case 890 for convenient transport.

The thermal barrier 882 and the phase change materials 884 can be used to insulate the system 800. The electronics module 886 can be electrically coupled to the perfusion system 800, such as to provide power, and allow connection of the system 800 to a user interface. The oxygen tank 888 can be fluidly connected to the system 800 and provide oxygen-rich gas for perfusion of tissue. The carry case 890 can allow for movement of the system 800, such as during organ transport.

A sterile bag or flexible enclosure 885 may be interposed between the perfusion system 800 and the thermal barrier 882, as shown in FIG. 8C. The sterile bag may be used to preserve sterility of the outer surfaces of the perfusion system 800 during transport within the thermal barrier 882 and outer case 890.

### Various Notes & Examples

1. A perfusion system includes a perfusate source and a perfusate distributor coupled to the perfusate source and configured to convey oxygen containing perfusate from the perfusate source to tissue and exhaust carbon dioxide generated by the tissue. A carbon dioxide sensor is coupled to sense the carbon dioxide generated by the tissue.
2. The perfusion system of example 1 and further comprising circuitry coupled to receive an indication from the carbon dioxide sensor, the indication representing a concentration of carbon dioxide.
3. The perfusion system of example 2 wherein the circuitry obtains a mass value representative of the mass of the tissue and a flow rate of fluid containing carbon dioxide flowing past the carbon dioxide sensor.
4. The perfusion system of example 3 wherein the circuitry is configured to generate a tissue viability indication based on the carbon dioxide data, the tissue mass value, tissue type and metabolic state, and the fluid flow rate.
5. The perfusion system of any of examples 1-4 wherein the perfusate distributor comprises a perfusate circulation system having a gas exchanger coupled to oxygenate the perfusate and to remove carbon dioxide from perfusate from the tissue and further comprising an exhaust conduit coupled to the gas exchanger to receive carbon dioxide removed from the carbon dioxide containing perfusate.
6. The perfusion system of example 5 wherein the carbon dioxide sensor is coupled to the exhaust conduit to measure carbon dioxide in the exhaust conduit.
7. The perfusion system of example 6 wherein the carbon dioxide is in gas form.
8. The perfusion system of any of examples 6-7 and further including an oxygen sensor coupled to the exhaust conduit to measure oxygen in the exhaust conduit.
9. The perfusion system of example 8 and further including circuitry coupled to an indication from the carbon dioxide sensor, the indication representing a concentration of carbon dioxide and an indication from the oxygen sensor representative of sensed oxygen concentration.
10. The perfusion system of example 9 wherein the circuitry is configured to compute a respiratory quotient from the carbon dioxide data and oxygen data.
11. The perfusion system of example 10 wherein the circuitry is configured to generate a tissue viability indication as a function of the respiratory quotient.
12. The perfusion system of any of examples 6-11 and further including a gas flow sensor coupled to measure gas flow between the oxygen source and the gas exchanger.
13. The perfusion system of any of examples 1-12 wherein the perfusate is a liquid.
14. The perfusion system of example 13 wherein the carbon dioxide sensor comprises a portion of the perfusate distributor and is disposed downstream of the tissue to measure carbon dioxide generated by the tissue in the perfusate.
15. The perfusion system of any of examples 1-14 and further including a pump coupled to move perfusate through the perfusate distributor.
16. The perfusion system of any of examples 1-15 and further including a tissue canister configured to receive the tissue, the tissue canister comprising ports for coupling to the perfusate distributor.
17. The perfusion system of any of examples 1-16 and further including a gas exchanger coupled to an oxygen source and the perfusate distributor and a gas flow regulator coupled between the oxygen source and the gas exchanger.
18. The perfusion system of example 17 wherein the gas flow regulator is to regulate the flow of oxygen to the gas exchanger.
19. The perfusion system of example 18 and further including circuitry coupled to control the gas regulator.
20. The perfusion system of any of examples 1-12 wherein the perfusate is a humidified gas.
21. A perfusion system includes a gas exchanger to receive oxygen from an oxygen source and a perfusate circulation system coupled to the gas exchanger and configured to convey oxygen containing perfusate from the gas exchanger to tissue and return carbon dioxide containing perfusate from the tissue to the gas exchanger. An exhaust conduit is coupled to the gas exchanger to receive gas containing carbon dioxide removed from the returned carbon dioxide containing perfusate. A carbon dioxide sensor is coupled to the exhaust conduit to measure carbon dioxide concentration in the carbon dioxide-containing gas. A controller is coupled to receive carbon dioxide indication from the carbon dioxide sensor to determine a rate of carbon dioxide generation by the tissue based on the carbon dioxide data, and a flow rate of the gas.
22. A perfusion system includes a gas exchanger to receive oxygen from an oxygen source and a perfusate circulation system coupled to the gas exchanger and configured to convey oxygen containing perfusate from the gas exchanger to tissue and return carbon dioxide containing perfusate from the tissue to the gas exchanger. A carbon dioxide sensor is coupled to measure carbon dioxide generated by the tissue. A controller is coupled to receive a carbon dioxide indication from the carbon dioxide sensor to determine a rate of carbon dioxide generation by the tissue based on the carbon dioxide concentration and flow rate of the gas.
23. A perfusion system includes a gas exchanger to receive oxygen from an oxygen source and a perfusate circulation system coupled to the gas exchanger and configured to convey oxygen containing perfusate from the gas exchanger to tissue and return carbon dioxide containing perfusate from the tissue to the gas exchanger. A carbon dioxide sensor is coupled to the perfusate circulation system downstream of the tissue to measure carbon dioxide generated by the tissue. A controller is coupled to receive carbon dioxide data from the carbon dioxide sensor to determine a rate of carbon dioxide generation by the tissue based on the carbon dioxide data, a mass of the tissue, and a flow rate of the gas.
23. A method includes perfusing tissue with an oxygenated perfusate, receiving perfusate from the tissue, measuring carbon dioxide generated by the tissue, and computing a measure of tissue viability based on the measured carbon dioxide, flow rate of the perfusate, and physical characteristics of the tissue.
24. The method of example 23 wherein measuring carbon dioxide generated by the tissue is performed by measuring a carbon dioxide concentration difference and multiplying the carbon dioxide concentration by a flow rate.
25. The method of example 23 wherein measuring carbon dioxide includes extracting carbon dioxide from the perfusate received from the tissue, flowing the extracted carbon dioxide past a carbon dioxide sensor, and receiving a value corresponding to carbon dioxide concentration in the extracted carbon dioxide.
26. The method of example 23 and further including measuring oxygen consumed by the tissue.
27. The method of example 26 and further comprising calculating a respiratory quotient from the measured carbon dioxide and oxygen.
28. A perfusion system includes a gas exchanger to receive oxygen from an oxygen source and a perfusate circulation system coupled to the gas exchanger and configured to convey oxygen containing perfusate from the gas exchanger to tissue and return carbon dioxide containing perfusate from the tissue to the gas exchanger. A carbon dioxide sensor is coupled to measure carbon dioxide generated by the tissue. Each of these non-limiting examples can stand on its own or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A perfusion system comprising:
a perfusate source;
a perfusate distributor coupled to the perfusate source and configured to convey oxygen containing perfusate from the perfusate source to tissue and exhaust carbon dioxide generated by the tissue; and
a carbon dioxide sensor coupled to sense the carbon dioxide generated by the tissue.

2. The perfusion system of claim 1 and further comprising circuitry coupled to receive an indication from the carbon dioxide sensor, the indication representing a concentration of carbon dioxide.

3. The perfusion system of claim 2 wherein the circuitry obtains a mass value representative of the mass of the tissue and a flow rate of fluid containing carbon dioxide flowing past the carbon dioxide sensor.

4. The perfusion system of claim 3 wherein the circuitry is configured to generate a tissue viability indication based on the carbon dioxide data, the tissue mass value, tissue type and metabolic state, and the fluid flow rate.

5. The perfusion system of claim 1 wherein the perfusion distributor comprises a perfusate circulation system having a gas exchanger coupled to oxygenate the perfusate and to remove carbon dioxide from perfusate from the tissue and further comprising an exhaust conduit coupled to the gas exchanger to receive carbon dioxide removed from the carbon dioxide containing perfusate.

6. The perfusion system of claim 5 wherein the carbon dioxide sensor is coupled to the exhaust conduit to measure carbon dioxide in the exhaust conduit.

7. The perfusion system of claim 6 wherein the carbon dioxide is in gas form.

8. The perfusion system of claim 6 and further comprising an oxygen sensor coupled to the exhaust conduit to measure oxygen in the exhaust conduit.

9. The perfusion system of claim 8 and further comprising circuitry coupled to an indication from the carbon dioxide sensor, the indication representing a concentration of carbon dioxide and an indication from the oxygen sensor representative of sensed oxygen concentration.

10. The perfusion system of claim 9 wherein the circuitry is configured to compute a respiratory quotient from the carbon dioxide data and oxygen data.

11. The perfusion system of claim 10 wherein the circuitry is configured to generate a tissue viability indication as a function of the respiratory quotient.

12. The perfusion system of claim 6 and further comprising a gas flow sensor coupled to measure gas flow between the oxygen source and the gas exchanger.

13. The perfusion system of claim 1 and further comprising a pump coupled to move perfusate through the perfusion distributor.

14. The perfusion system of claim 1 and further comprising a tissue canister configured to receive the tissue, the tissue canister comprising ports for coupling to the perfusion distributor.

15. The perfusion system of claim 1 and further comprising:
a gas exchanger coupled to an oxygen source and the perfusion distributor; and
a gas flow regulator coupled between the oxygen source and the gas exchanger.

16. The perfusion system of claim 15 wherein the gas flow regulator is to regulate the flow of oxygen to the gas exchanger.

17. A perfusion system comprising:
a gas exchanger to receive oxygen from an oxygen source;
a perfusate circulation system coupled to the gas exchanger and configured to convey oxygen containing perfusate from the gas exchanger to tissue and return carbon dioxide containing perfusate from the tissue to the gas exchanger;
an exhaust conduit coupled to the gas exchanger to receive gas containing carbon dioxide removed from the returned carbon dioxide containing perfusate;
a carbon dioxide sensor coupled to the exhaust conduit to measure carbon dioxide concentration in the carbon dioxide-containing gas; and
a controller coupled to receive carbon dioxide indication from the carbon dioxide sensor to determine a rate of carbon dioxide generation by the tissue based on the carbon dioxide data, and a flow rate of the gas.

18. A method comprising:
perfusing tissue with an oxygenated perfusate;
receiving perfusate from the tissue;
measuring carbon dioxide generated by the tissue; and
computing a measure of tissue viability based on the measured carbon dioxide, flow rate of the perfusate, and physical characteristics of the tissue.

19. The method of claim 18 wherein measuring carbon dioxide generated by the tissue is performed by:
measuring a carbon dioxide concentration difference; and
multiplying the carbon dioxide concentration by a flow rate.

20. The method of claim 18 wherein measuring carbon dioxide comprises:
extracting carbon dioxide from the perfusate received from the tissue;
flowing the extracted carbon dioxide past a carbon dioxide sensor; and
receiving a value corresponding to carbon dioxide concentration in the extracted carbon dioxide.
